(19) United States
(12) Patent Application Publication
(10) Pub. No.: US 2006/0217797 A1
(43) Pub. Date: Sep. 28, 2006

(54) Asymmetric drug eluting hemodialysis graft

(76) Inventor: Samuel J. Wong, Piedmont, CA (US)

Correspondence Address: KUDIRKA & JOBSE, LLP, ONE STATE STREET, SUITE 800, BOSTON, MA, 02109, US

(21) Appl. No.: 11/149,781

(22) Filed: Jun. 10, 2005

Related U.S. Application Data
(63) Continuation-in-part of application No. 10/443,722, filed on May 23, 2003, now abandoned.

(51) Int. Cl. A61F 2/06

(52) U.S. Cl. 623/1.42; 427/2.21

(57) ABSTRACT

A hemodialysis graft has one end provided with a deposit containing an anti-stenotic agent. The coated end is connected to a vein to provide protection against stenosis occurring at the venous anastomosis.

### RELATED APPLICATIONS

This application is a continuation-in-part of non-provisional patent application Ser. No. 10/443,722 filed May 23, 2003, published as US2003/0029392 on Dec. 11, 2003, the entirety of which is incorporated by reference herein.

### FIELD OF THE INVENTION

The present invention relates to a hemodialysis graft that provides controlled and sustained delivery of an anti-stenotic agent to prevent stenosis in and around the hemodialysis graft when the graft is positioned in a patient.

### BACKGROUND OF THE INVENTION

A graft, in one known usage, is a medical device that is used as an artificial conduit for bodily fluids. A vascular graft provides a conduit for blood. When used in conjunction with hemodialysis, the vascular graft serves as a nonstatic reservoir of blood that is readily accessible by a dialysis machine. In many ways, the vascular graft serves as a lifeline, i.e., an essential interface between the patient and the dialysis machine.

Hemodialysis is not possible without access to the vascular system to provide an adequate and reliable source of blood to the hemodialysis machine. Currently, the most common type of vascular access used for dialysis in the United States is an artificial vascular graft 100, as shown in FIG. 1A, usually composed of expanded polytetrafluoroethylene (ePTFE) or as available from, for example, W.L. Gore & Associates, Flagstaff, Ariz. The artificial vascular graft 100 is typically tubular and has a predetermined length L. At a first end of the graft 100, an inner diameter d₁ is provided and at the opposing end an inner diameter d₂ is provided, as shown in FIGS. 1B-1D. As is known, the artificial graft 100 may have a constant inner diameter where d₁=d₂. Further, it is known to provide an artificial graft 100 with an inner diameter that varies where, for example, the inner diameter increases from one end to another and thus, for example, d₂>d₁.

As shown in FIG. 2, a hemodialysis graft 100 is inserted subcutaneously underneath the dermis of an extremity 200. One end of the graft 100 is attached to a feeding artery 202 and the other end is attached to a draining vein 204. Blood is diverted from the feeding artery 202, into the graft 100, and empties into the draining vein 204. Needles (not shown) are inserted percutaneously into the lumen of the graft 100 thereby allowing blood to be accessed for dialysis. Also as shown in FIG. 2, blood in the artery 202 flows in an arterial flow direction 206 and, in the vein 204, the blood flows in a venous flow direction 208. As the graft 100 connects the artery 202 to the vein 204, at an arterial anastomosis 210 and a venous anastomosis 212, respectively, blood in the graft 100 flows in a graft flow direction 214.

Stenosis at the graft-vein junction (venous anastomosis 212) is problematic as it commonly occurs and accounts for a majority of graft failures. Stenosis at the venous anastomosis 212 is due to neointimal hyperplasia, a result of endovascular injury when the graft 100 is attached to the vein 204. Through a complex process mediated by the immune system, a layer of scar-like material known as venous intimal hyperplasia develops within the lumen of the graft 100 and locally around the draining vein 204 near the venous anastomosis 212. Neointimal hyperplasia appears to develop regardless of whether the graft 100 is composed of ePTFE, Dacron, or bovine carotid artery. It has been reported that neointimal hyperplasia occurs predominately at both a heel of the graft 100 and at a floor of the draining vein 204. Endothelialization of the lumen of the graft 100 apparently does not occur.

Venous intimal hyperplasia is characterized by: (1) presence of smooth muscle cells/myofibroblasts, (2) an accumulation of extracellular matrix components, (3) angiogenesis within the neointima and adventitia, and (4) presence of an active macrophage cell layer lining the ePTFE material. Platelet-derived growth factor (PDGF), basic fibroblast growth factor (bFGF), and vascular endothelial growth factor (VEGF) are expressed by smooth muscle cells/myofibroblasts.

It has also been reported that animal models mimic the experience with humans. In a sheep model where an ePTFE graft was attached to the carotid artery at one end and the other end attached to the external jugular vein, a layer of venous intimal hyperplasia was present by four weeks and was significantly thicker at the venous anastomosis compared to the arterial end by eight weeks.

Currently, the main treatment for stenosis at the venous anastomosis is either surgical revision of the venous anastomosis or percutaneous transluminal angioplasty (PTA) of the venous anastomosis. In surgical revision of the venous anastomosis, the stenotic graft segment is replaced with a new segment of ePTFE or the venous anastomosis is cut open, making the anastomosis wider, and a patch of ePTFE is then over sewn (patch angioplasty). In PTA, a balloon is deployed endovascularly across the stenosis and inflated; breaking up the stenotic area. Restenosis is common and the medium and long term patency rates are poor. Patency rates diminish with subsequent interventions. As one might expect, these measures treat the immediate problem but do not address the underlying pathology and neointimal hyperplasia will reoccur eventually, resulting in graft loss.

Bare metal stents have been tried to maintain patency at the venous anastomosis but their patency rates have been disappointing. Wallstents were found to have a shorter patency period compared to PTA, while Gianturco Z stents did not fare much better having an equivalent patency to PTA.

Drug eluted stents (DES) have had excellent results in the treatment of coronary artery disease, but as of yet, no studies have been performed for the treatment of venous intimal hyperplasia in hemodialysis grafts.

Studies looking at the prevention of venous intimal hyperplasia using systemically administered heparin and low molecular weight dextran did not find any statistical difference in the attenuation of venous intimal hyperplasia after three months.

Several studies using local external beam or endovascular radiation (brachytherapy) demonstrated a significant reduction in neointimal hyperplasia compared to control in an arteriovenous ePTFE graft. The study using external beam radiation, however, was in a pig model and only twelve pigs were used and the ePTFE was excised after only twenty-eight days. Another study used external beam radiation in a canine model with the ePTFE graft excised over a much long period of time, three and six months, but only used eight dogs. Endovascular radiation yielded similar results in a pig model with an ePTFE graft excised only after six weeks where only eight pigs were used. Long term attenuation of venous intimal hyperplasia over one year was not studied.

### SUMMARY OF THE INVENTION

An asymmetric hemodialysis graft has at least one coating deposit at the end to which the patient's vein is attached. The coating deposit includes a polymer having a therapeutic dosage of an agent, for example, an anti-stenotic, that is released over a period of time to reduce the occurrence of stenosis at the graft-vein junction. A plurality of coating deposits may be provided where each includes different therapeutic agents or amounts that different from another deposit. Advantageously, providing the anti-stenotic directly at the location where stenosis most frequently occurs directs the therapeutic agent most efficiently and requires a minimal amount of material.

In one embodiment of the present invention, a hemodialysis graft comprises a cylindrical tube having a lumen therethrough, a first open end and an opposed second open end, the tube having an interior surface and an exterior surface; a first coating deposit located at the first open end of the tube and extending toward the second open end along the interior surface a first predetermined distance; and a second coating deposit located at the first open end of the tube and extending toward the second open end along the exterior surface a second predetermined distance. The first coating deposit comprises a first therapeutic agent and the second coating deposit comprises a second therapeutic agent.

In another embodiment, a graft comprises: a substantially cylindrical and hollow tube of a predetermined longitudinal length having a distal end with a distal opening, a proximal end with a proximal opening, an interior surface and an exterior surface; a first coating deposit located substantially only on the interior surface at the distal end and extending proximally and longitudinally a first predetermined distance; and a second coating deposit located substantially only on the exterior surface at the distal end and extending proximally and longitudinally a second predetermined distance. The first coating deposit comprises a first therapeutic agent and the second coating deposit comprises a second therapeutic agent, and the tube comprises a substantially impermeable material.

### BRIEF DESCRIPTION OF THE DRAWINGS

The above and further advantages of the invention may be better understood by referring to the following description in conjunction with the accompanying drawings in which:

FIGS. 1A-1D are schematic and perspective views of an artificial graft;

FIG. 2 is a perspective view of a placement of an artificial graft in a patient's extremity;

FIG. 3 is a schematic diagram of a venous anastomosis;

FIG. 4 is an asymmetric graft, according to one embodiment of the present invention;

FIG. 5 is an asymmetric graft according to another embodiment of the present invention;

FIG. 6 is an asymmetric graft according to another embodiment of the present invention;

FIG. 7 is a cross sectional view of a drug eluting graft;

FIG. 8 is a cross sectional view of a drug eluting graft;

FIG. 9 is a flow chart of a method of making an asymmetric graft, according to one embodiment of the present invention; and

FIG. 10 is a schematic drawing of one embodiment of an asymmetric drug eluting graft according to the present invention.

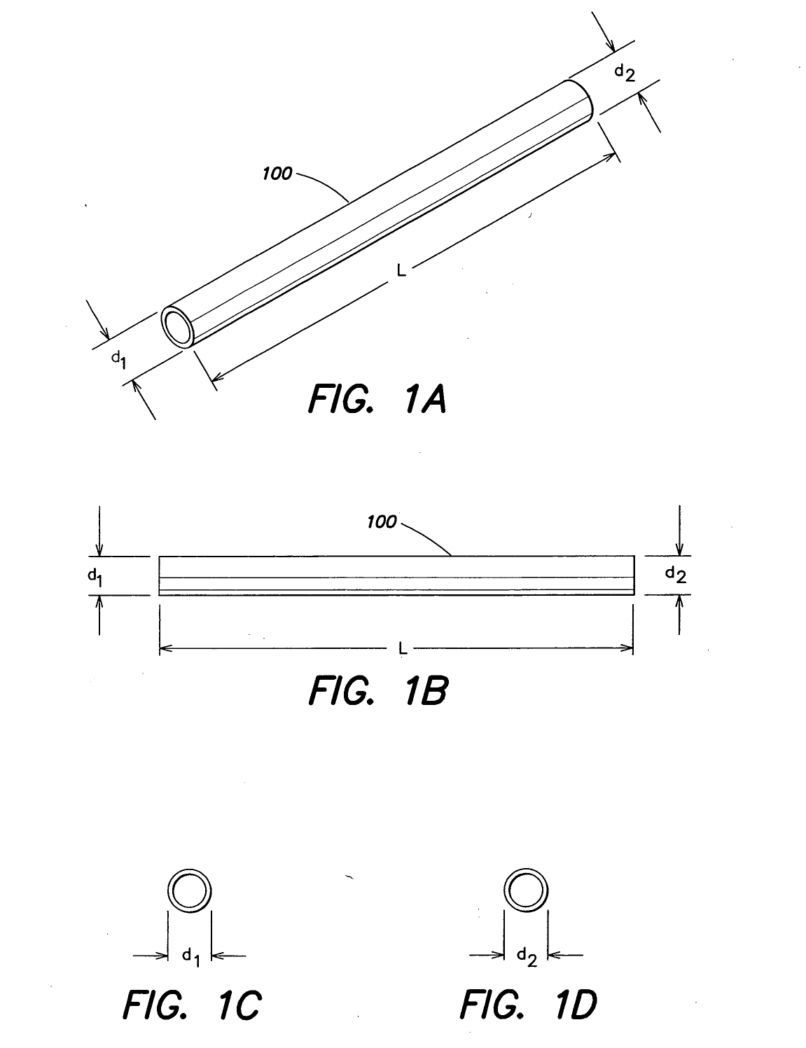

### DETAILED DESCRIPTION

Neointimal hyperplasia occurs within four weeks of insertion of a hemodialysis graft 100. While neointimal hyperplasia is seen along the entire hemodialysis graft 100, severe or flow limiting venous intimal hyperplasia occurs predominately at the venous anastomosis 212. Hemodynamic factors unique to artificial hemodialysis grafts appear to contribute to the rapid development of neointimal hyperplasia at the venous anastomosis 212. Smooth muscle cells, extracellular matrix and macrophages contribute to the formation of this venous intimal hyperplasia. Thus, specific agents targeting these cellular elements may be beneficial.

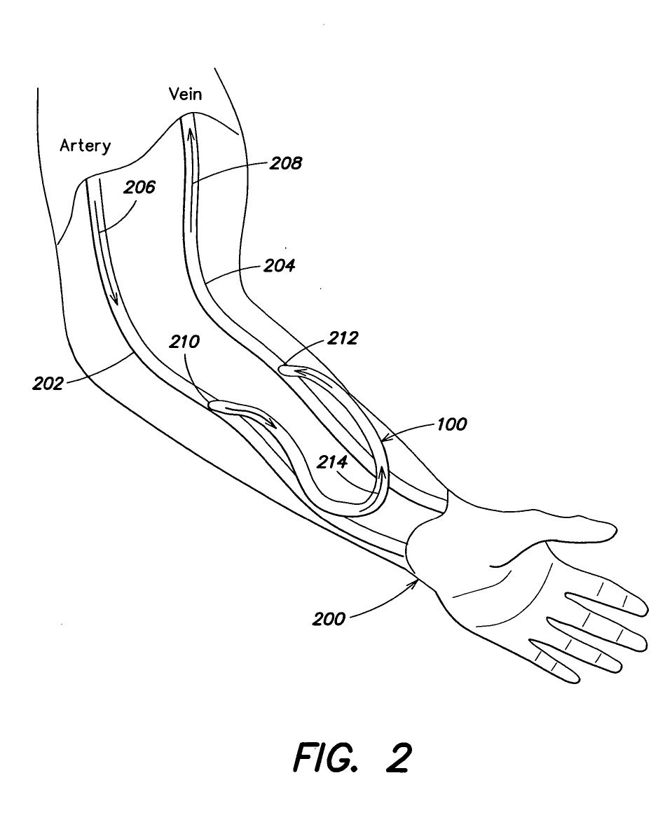

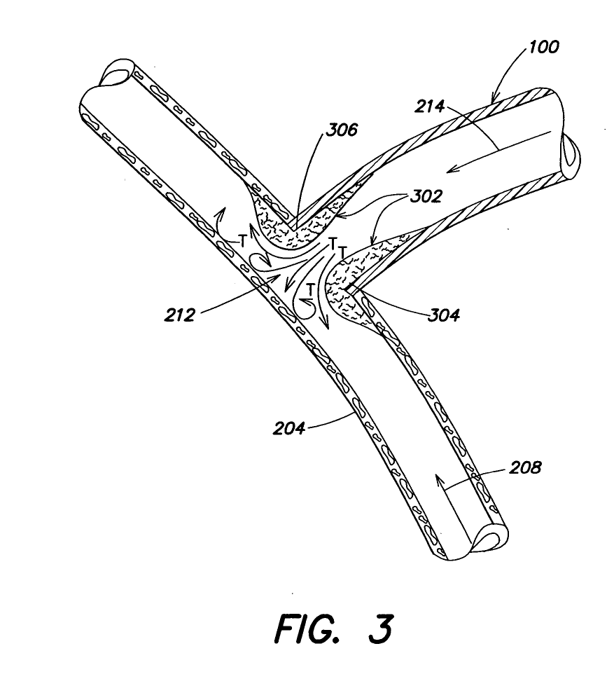

Why significantly flow limiting venous intimal hyperplasia disproportionately occurs at the venous anastomosis 212 in a hemodialysis graft 100 compared to other parts of the vascular graft 100 appears to be due to hemodynamic factors at the venous anastomosis 212 influencing the formation of neointimal hyperplasia. As shown in FIG. 3, a portion of FIG. 2, one end of the graft 100 is attached to the draining vein 204 at the venous anastomosis 212. There is a tremendous pressure differential from a high pressure system, i.e., the artery 202, to a low pressure system, i.e., the vein 204, and turbulent flow T occurs within the venous anastomosis 212. The vascular endothelium is damaged, leading to the formation of venous intimal hyperplasia 302 at a heel 304 and a toe 306 of the venous anastomosis 212.

This situation differs from arterial bypass grafts where one end is attached to an artery and the other end is also attached to an artery; a situation seen in coronary artery bypass (CABG) although the graft is usually harvested from the saphenous vein. Low shear stress and oscillating shear forces at the arterial floor plus a high wall sheer stress at the venous anastomosis probably promote neointimal hyperplasia development. Compliance mismatch between the graft 100 and the artery 202 causes turbulence that may also contribute. The blood flow rate in a graft is 5-10 times greater than in arterial bypass grafts. The high flow causes turbulence that injures endothelial cells and eventually results in the neointimal hyperplasia 302.

The following will describe, in more detail, a hemodialysis graft according to different embodiments of the present invention that inhibits the development of venous intimal hyperplasia at the venous anastomosis.

As will be described, the hemodialysis graft contains:

1) An asymmetric deposit of one or more anti-stenotic agents at the venous end of the hemodialysis graft.

2) The deposit of anti-stenotic agents coats the exterior of the hemodialysis graft at the end of the graft to which the vein is to be attached. The anti-stenotic agents diffuse locally to prevent neointimal hyperplasia from developing at the floor of the draining vein near the venous anastomosis.

3) The deposit of anti-stenotic agents coats the interior of the hemodialysis graft at the end of the graft to which the vein is to be attached. The anti-stenotic(s) agent diffuse within the interior lumen to prevent neointimal hyperplasia from developing in the interior lumen of the graft. A deposit of antistenotic agents located in the interior of the graft is advantageous because the graft is oftentimes composed of material, such as ePTFE, that is impermeable to diffusion of ant-stenotic agents from the exterior into the interior and vice versa. In addition, any antistenotic agents that did diffuse across the permeable vein from the deposit of anti-stenotic agents coating the exterior end the graft could not diffuse upstream into the interior lumen of the graft.

4) A single bioerodible polymer, or a mixture of two or more bioerodible polymers, may be used. Sustained and controlled local delivery of the anti-stenotic agents may be achieved without the toxic side effects from the antistenotic agent(s) when given systemically at doses required to achieve the antistenotic effect. A biodegradable polymer that releases the anti-stenotic agents over weeks or months would be advantageous because neointimal hyperplasia develops rapidly after graft implantation.

5) The biodegradable polymer may be a polylactic acid, a polyglycolic acid, a polycaprolactone, a polyhydroxybutyrate, a polyhydroxyvalerate, a polyanhydride, a polyorthoester, a poly (amino acids), a psuedopolyamino acid, or a polyphosphazene.

6) Alternatively, a single nonbiodegradable polymer, or a mixture of two or more nonbiodegradable polymers, may be used. The nonbiodegradable polymer may be a silicone, poly (ethylene vinyl acetate), a poly (methyl methacrylate), polyethylene, polyurethane, polyisobutylene, cellulose acetate, poly (ethyl methacrylate), poly (butyl methacrylate). A nonbiodegradable polymer is able to provide prolonged local delivery of anti-stenotic agents to prevent neointimal hyperplasia from developing over a long term, e.g., potentially more than one year.

7) Smooth muscle cells are intimately involved in the formation of neointimal hyperplasia. The anti-stenotic agents may be a taxane such as paclitaxel or its derivatives, an antiproliferative agent such as cyclophosphamide, methotrexate, and the like.

8) Macrophages are also involved in the formation of neointimal hyperplasia. The anti-stenotic agents may be a steroid such as methylprednisolone or its derivatives, or an FKBP binder such as sirolimus, cyclosporine, or tacrolimus.

For simplicity and ease of explanation, the following will reference only one ant-stenotic agent and the antistenotic agent will be the same for each example. As one of ordinary skill in the art will understand, a different anti-stenotic agent or more than one anti-stenotic agent may be used in combination in accordance with the present invention. Differences in drug carriers used will be highlighted.

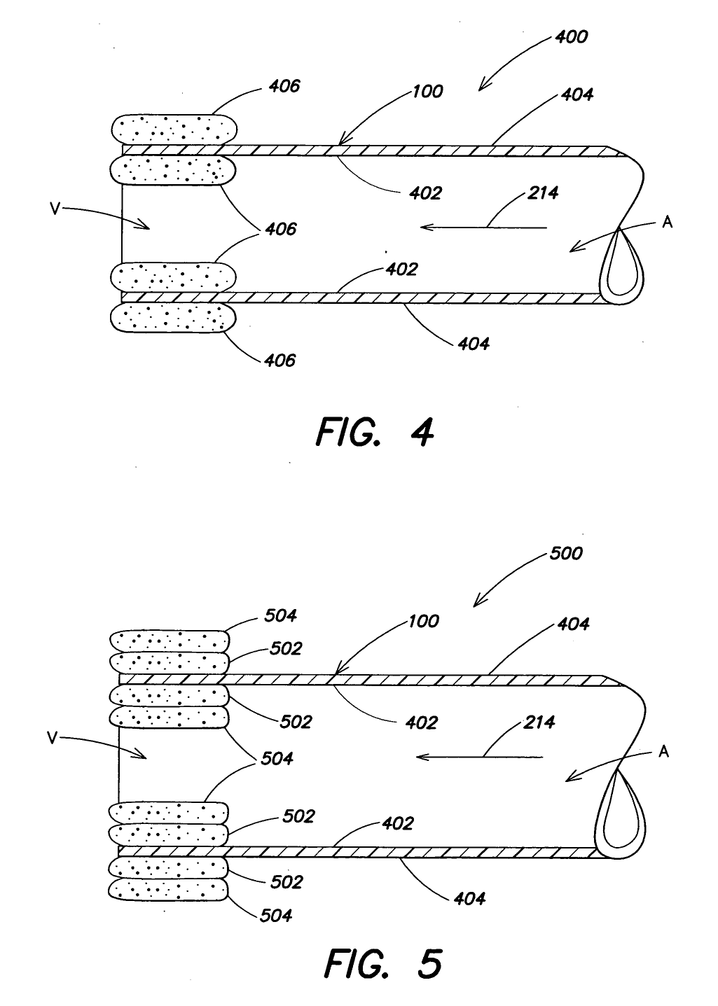

According to one embodiment of the present invention, as shown in FIG. 4, an asymmetric graft 400 is provided. The asymmetric graft 400 is based on a known graft 100 having an inner, or luminal, surface 402 and an exterior surface 404. For ease of explanation and orientation, the asymmetric graft 400 is provided with a venous end V and an arterial end A. Thus, the graft flow direction 214 is from the arterial end A to the venous end V.

In one embodiment, a solution of 30% paclitaxel by weight is mixed in a solution of poly (lactic acid) with a molecular weight of 20,000 to 30,000 Daltons. Using conventional techniques, this mixture is applied, e.g., by spraying or dipping, to the venous end V of the graft 100 to provide a coating deposit 406. Both the exterior 404 and the interior surface 402 of the venous end V of the graft 100 would be coated to form the coating deposit 406 of anti-stenotic agent available for local delivery at the venous end V of the asymmetric graft 400. The length of the coating deposit 406, in one embodiment, is 1 to 3 cm in length, nominally 2 cm, from the venous end V and the thickness is in the range of 0.01 to 1 mm.

In an alternate embodiment, the coating deposit 406 may be comprised of a solution of 30% paclitaxel by molecular weight mixed in a solution of nonbiodegradable polymer such as poly (ethylene vinyl acetate) 30% by weight, commercially available from the Alza Corporation, Mountain View, Calif. The venous end V of the graft 400 is dipped or sprayed in the conventional manner, coating the exterior is surface 404 and the interior surface 402 of the dialysis graft 400. The length of the coating deposit 406 is 1 to 3 cm in length, measured from the venous end V with a thickness in the range of 0.01 mm to 1 mm. Sustained release of an antistenotic agent of over a period of years may be potentially favorable if neointimal hyperplasia that would otherwise be suppressed in the intermediate term (months) returns in the long term (years).

Alternatively, with respect to the graft flow direction 214, the arterial end A may be considered a proximal end with the venous end V considered a distal end of the asymmetric graft 400. Thus, the positioning of the coating deposit 406 may be described as located at the distal end of the asymmetric graft 400 and extending proximally in a longitudinal direction along the interior surface 402 and the exterior surface 404.

As shown in FIG. 5, a multilayer asymmetric graft 500, according to one embodiment of the present invention, is depicted. Similar to the asymmetric graft 400 shown in FIG. 4, the multilayer asymmetric graft 500 may be based on a conventional graft 100 as well.

In one embodiment, a solution of 30% paclitaxel by weight is mixed with a solution of nonbiodegradable polymer such as poly (ethylene vinyl acetate) 30% by weight. The venous end V of the graft 400 is dipped or sprayed in the conventional manner, coating the exterior surface 404 and the interior surface 402 of the graft 400 to provide an undercoat deposit 502. The length of the undercoat deposit 502 would be 1 to 3 cm from the venous end V with a thickness in the range of 0.01 to 1 mm. A top coat deposit 504, made from a mixture of 30% paclitaxel and a solution of poly (lactic acid) with a molecular weight of 20,000 to 30,000 Daltons, is dipped or sprayed at the venous end V of the graft 400 over the undercoat deposit 502. The length of the topcoat deposit 504 from the venous end V would be in the range of 1 to 3 cm with a depth of 0.01 to 1 mm. The top coat deposit 504, would have the advantage of attenuating any neointimal hyperplasia 302 from developing in the short (weeks) and intermediate term (months) and the undercoat deposit 502 would attenuate any neointimal hyperplasia 302 from developing in the long term (years).

The multilayer graft 500 has been shown with two layers 502, 504 for explanatory purposes only. It is envisioned that more than two layers may be provided and such an embodiment is considered part of the present invention.

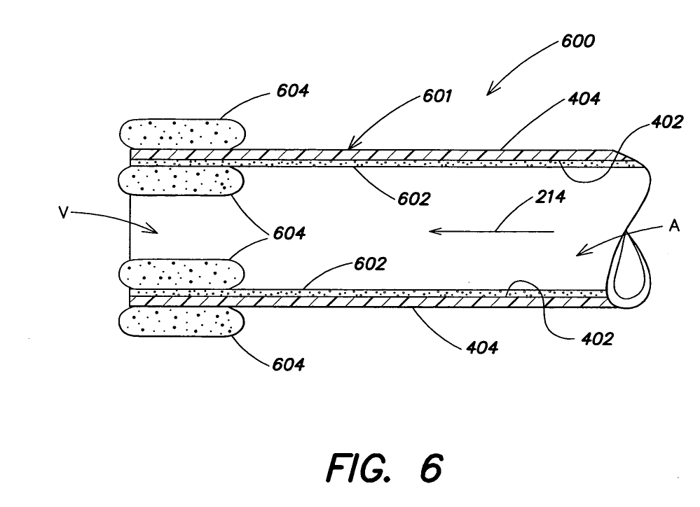

In accordance with another embodiment of the present invention, a drug eluting asymmetric graft 600 is provided, as shown in FIG. 6. The asymmetric drug eluting graft 600 is a modification to a drug eluting graft 601 as described in currently pending U.S. patent application Ser. No. 10/443,722, filed May 23, 2003, published as US2003/0229392 on Dec. 11, 2003, and assigned to the assignee of the present application, the entire contents of which are incorporated herein in their entirety. The drug eluting graft 601 includes a drug eluting layer 602 covering substantially the entire interior lumen surface 402 of the drug eluting graft 601. The drug eluting layer 602, in one embodiment, comprises at least one therapeutic agent in a nonbioerodable polymer.

The drug eluting layer 602 may include at least one therapeutic agent, e.g., an antiproliferative agent such as sirolimus and paclitaxel. Other antiproliferative agents such as cyclophosphamide, actinomycin D, mitomycin, steroid, angiotensin inhibitor, nitric oxide donor, calcium channel blocker, anti-sense nucleic acid, thiazolidinedione, or HMG Co A reductase inhibitor may afford similar results.

In one embodiment, the at least one therapeutic agent in the drug eluting layer 602 is in microcapsules dispersed within the polymer. Further, the microcapsules can have a wall formed of a drug release rate controlled material so that the therapeutic agent within the microcapsule is released in a controlled and continuous rate over a prolonged period of time.

In another embodiment, the at least one therapeutic agent disposed in the drug eluting layer 602 is selected from a group consisting of: a) an antimicrotuble agent such as paclictaxel, docetaxel; b) an antiproliferative agent such as cyclophosphamide, actinomycin-D, cis-platinum, mitomycin, methotrexate, azithioprim; c) an immunosupressive agent such as sirolimus, tacrolimus, cyclosporine A, or a steroid such as dexamethasone or methylprednisolone; d) a glycoprotein IIb/IIIa receptor inhibitor such as abciximab, eptifibatide, tirofiban, sibrafiban, xemilofiban, orbofiban, roxifiban, lotrabian; e) a platelet aggregation inhibitor such as clopidogrel or ticlopidine; f) a nitric oxide donor such as nitroglycerin, isosorbide dinitrate, or ntiroprusside; g) a calcium channel blocker (verapamil, diltiazem, nifedipine, etc.); h) an antithrombogenic agent (heparin, low molecular weight heparin, hirudin); i) an anti-sense nucleic acid; j) a thiazolidinedione; k) an HMG Co A reductase inhibitor such as pravastatin; I) an angiotensin converting enzyme inhibitor; and m) an omega 3 fish oil.

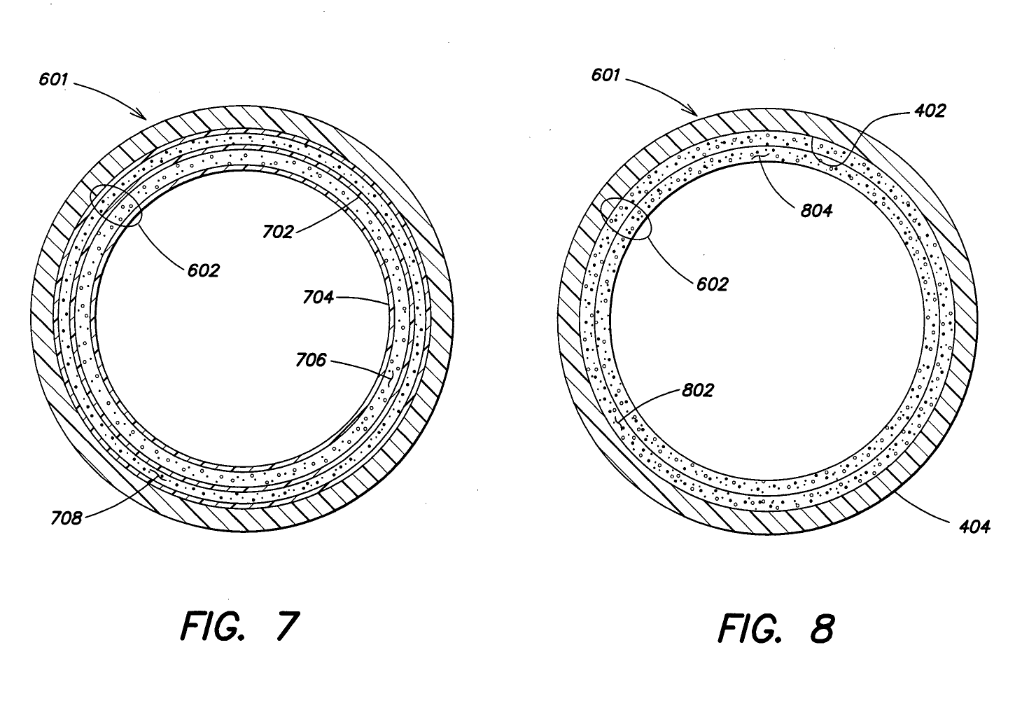

In another embodiment, the drug eluting layer 602 comprises at least one erodible polymer and at least one therapeutic agent. Further, the drug eluting layer 602 may be comprised of two or more layers of polymer(s) 702, 704 with the therapeutic agent 706, 708 sandwiched in between each layer as shown in FIG. 7 or different layers 802, 804 of a mixture of polymer/therapeutic agent(s) as shown in FIG. 8.

The polymer, therapeutic agent or mixture of polymer/therapeutic agents may be applied using conventional dip-coating or spray coating techniques where the polymer and therapeutic agent may be suspended in an organic solvent. The solvent then evaporates leaving a coat/layer of polymer or therapeutic agent.

The coating may also be achieved by vapor deposition, plasma polymerization or using an air suspension process as described in U.S. Pat. No. 6,368,658 to Schwarz et al.

Alternatively, layers of polymer and therapeutic agents can be achieved using electron beam deposition, electron beam polymerization or electron beam treatment process.

In one embodiment of the drug eluting asymmetric graft 600, a solution of 30% paclitaxel by weight is mixed in a solution of poly (lactic acid) with a molecular weight of 20,000 to 30,000 Daltons. Using conventional techniques, this mixture is applied by, e.g., spraying or dipping, to the venous end V of the drug eluting hemodialysis graft 601 to provide a coating deposit 604. In one embodiment, the drug eluted hemodialysis graft 601 would have a drug eluting layer 602 of 30% paclitaxel encased in a matrix of nonbioerodable polymer. The nonbiodegradable polymer may be silicone, poly(ethylene vinyl acetate), poly(methyl methacrylate), polyethylene, polyurethane, polyisobutylene, cellulose acetate, poly(ethyl methacrylate), poly(butyl methacrylate).

An advantage of the asymmetric drug eluting hemodialysis graft 600 is that not only would it inhibit neointimal hyperplasia 302 from developing at the venous anastomosis 212, but it would inhibit neointimal hyperplasia 302 from developing within a mid region of the graft 600 and at the arterial anastomosis 210. Significant flow limiting stenosis caused by neointimal hyperplasia 302 does not often occur at the arterial anastomosis 210 less than one year after implantation.

Flow limiting stenosis that may occur after years of forming neointimal hyperplasia 302 is usually not seen due to graft loss from neointimal hyperplasia 302 at the venous anastomosis 212. An asymmetric hemodialysis graft with a deposit of anti-stenotic agent located at the venous end V lasts many years and the neointimal hyperplasia at the arterial end A may then be more problematic. Stenosis in the mid graft is usually not a frequent problem unless it is left to develop over years. Stenosis of the mid graft in the short and intermediate term is usually a consequence of repeated needle sticks at the same segment of the graft leading to inflammation and accelerated neointimal hyperplasia. An antistenotic agent encased in a matrix of nonbiodegradable polymer would slowly release antistenotic agent potentially over years and may inhibit significant neointimal hyperplasia from developing in the arterial anastomosis and at the mid graft.

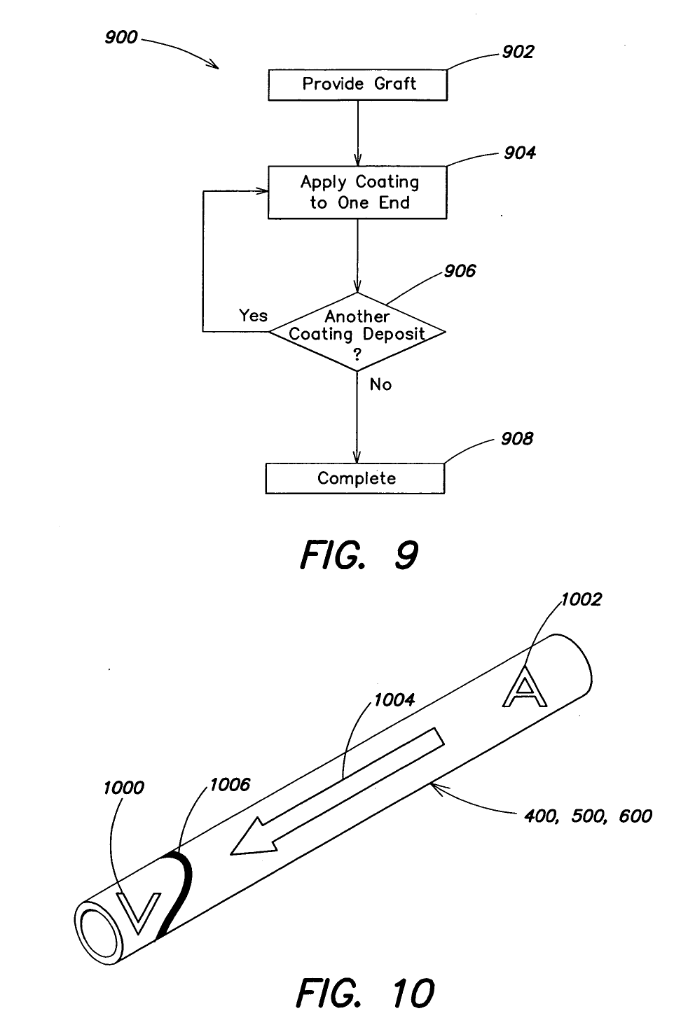

Referring to FIG. 9, a method 900 for creating the asymmetric grafts as described previously will now be discussed. In an initial step 902, a graft is provided. The provided graft may be either the artificial hemodialysis graft 100 as described above or the drug eluting graft 601. A coating is applied to one end of the graft, i.e., the venous end V, also referred to as the distal end. Next, step 906, if multiple deposits of coating are to be applied, then control returns to step 904 and the next coating deposit is applied. Alternately, if all coating deposits have been applied, control passes to the completion step 908.

In another embodiment, as shown in FIG. 10, any one of the asymmetric grafts 400, 500, 600 may be provided with markings to identify one or more of the venous end V the arterial end A or the graft flow direction 214. As shown in FIG. 10, a first marker 1000 identifies the venous end V of the graft. The first marker 1000 may be imprinted on the graft by any one of a number of methods known to those of skill in the art. In addition, the first marker 1000 may be placed circumferentially around the graft such that the respective end can be identified from any orientation of the graft. Similarly, a second marker 1002 identifies the arterial end A while a third marker 1004 represents the flow direction within the graft. These markers may be used by the physician when inserting the graft to ensure that the graft is properly placed in the patient.

In an alternative embodiment, the venous end V of any one of the foregoing embodiments of the asymmetric graft may be provided with a larger interior diameter than the arterial end A. An asymmetric graft with a larger inner diameter at its venous end V may compensate for the coating layers that are provided at the venous end V.

The graft may be made from any one or more of the following materials: expanded polytetrafluoroethylene (ePTFE); polyvinylchloride polypropylene; fluorinated ethylene propylene; polyetherurethaneurea ; and a biocompatible plastics material. One of ordinary skill in the art will understand that there may be other substantially impermeable materials from which the graft may be made. The foregoing list is not meant to be exhaustive nor limiting.

It is known that, in practice, a physician will trim the end of a graft that is being attached to the vein. This trimming is often done at an angle to the longitudinal axis of the graft so that the angle at which the graft is attached to the vein is less acute, i.e., less than 90°. By cutting at an angle, a larger cross-sectional area of the venous end of the graft is provided along with potentially less turbulence in the blood flow. Typically, this angle is in the range of 30° to 45°.

An exemplary length for the coating deposit was given in the range of 1 to 3 cm in the foregoing embodiments This range is not meant to be limiting and one of ordinary skill in the art will understand that a sufficient length of coating deposit will be provided to account for possible “trimming” of the graft. Nominally, for all embodiments described above, a length of coating of at least 2 cm should remain when inserted into a patient. Further, in another embodiment, a visual marker 1006 , such as the line shown in FIG. 10, may be provided to indicate the proximal end of the coating deposit so as to provide the physician with a visual aid when inserting the graft. This line could also be provided at an angle, e.g., 30° or 45° to give the physician a guide for cutting. Of course, the graft could be provided with an already angled venous end that would avoid the need for cutting by the physician altogether.

A recent small study has shown that a diet rich in ω-3 (omega-3) fatty acids derived from fish oil was beneficial in maintaining patency of a hemodialysis graft as compared to a control oil (placebo.) It has been suggested also that ω-3 fatty acids inhibit intimal hyperplasia in autogenous vein grafts. In one embodiment of the present invention, ω-3 fatty acids, or the active ingredients therein, is provided in at least one of the coating deposits for release over an extended period of time.

The foregoing embodiments have shown the coating deposits located at the venous end V, also referred to as the distal end, and extending proximally, i.e., toward the arterial end A, a same length on the interior surface and the exterior surface. In an alternate embodiment, a length which the interior coating deposit extends distally may be different from a length which the coating deposit on the exterior extends distally. Thus, an asymmetric graft according to one embodiment of the present invention may have different lengths of coating deposited as between the interior and exterior surfaces. Further, with respect to the multilayer asymmetric graft embodiment described above, layers of coating may extend distally at different lengths than the underlying layer and these distal lengths may differ as between the interior and exterior surfaces. One of ordinary skill in the art would understand how different lengths of coating deposit may be applied by operation of dipping or spraying, or combinations thereof in conjunction with masking or blocking techniques.

Other embodiments of the present invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A hemodialysis graft, comprising:
a cylindrical tube having a lumen therethrough, a first open end and an opposed second open end, the tube having an interior surface and an exterior surface;
a first coating deposit located at the first open end of the tube and extending toward the second open end along the interior surface a first predetermined distance; and
a second coating deposit located at the first open end of the tube and extending toward the second open end along the exterior surface a second predetermined distance;
wherein the first coating deposit comprises a first therapeutic agent and the second coating deposit comprises a second therapeutic agent.

2. The hemodialysis graft of claim 1, wherein at least one of the first and second therapeutic agents has anti-stenotic properties.

3. The hemodialysis graft of claim 1, wherein:
the first therapeutic agent is one of: paclitaxel, a paclitaxel derivative, sirolimus, an Interleukin-2 inhibitor, dexamethasone, a steroid, dopidogel, a platelet aggregate inhibitor, and fish oil; and
the second therapeutic agent is one of paclitaxel, a paclitaxel derivative, sirolimus, an Interleukin-2 inhibitor, dexamethasone, a steroid, dopidogel, a platelet aggregate inhibitor, and fish oil.

4. The hemodialysis graft of claim 1, wherein at least one of the first coating deposit and the second coating deposit are placed on the tube by at least one of dipping and spraying.

5. The hemodialysis graft of claim 1, wherein the first predetermined distance and the second predetermined distance are not substantially the same.

6. The hemodialysis graft of claim 1, wherein the interior surface comprises a drug eluting layer, the drug eluting layer comprising at least one bio-erodible polymer and at least one therapeutic agent.

7. The hemodialysis graft of claim 1, wherein the cylindrical tube comprises a substantially impermeable material.

8. The hemodialysis graft of claim 1, wherein the cylindrical tube comprises at least one of:
expanded polytetrafluoroethylene (ePTFE);
polyvinylchloride polypropylene;
fluorinated ethylene propylene;
polyetherurethaneurea; and
a biocompatible plastics material.

9. The hemodialysis graft of claim 1, wherein at least one of the first coating deposit and the second coating deposit comprises a bioerodible polymer.

10. The hemodialysis graft of claim 9, wherein:
each of a first amount of the first therapeutic agent and a second amount of the second therapeutic agent is chosen to elute a therapeutic dosage of the respective therapeutic agent over a period of time.

11. The hemodialysis graft of claim 10, wherein:
each of the first coating deposit and the second coating deposit comprises a bioerodible polymer in which the first therapeutic agent and the second therapeutic agent is mixed, respectively.

12. The hemodialysis graft of claim 1, wherein at least one of the first coating deposit and the second coating deposit comprises a non-bioerodible polymer.

13. The hemodialysis graft of claim 12, wherein:
each of a first amount of the first therapeutic agent and a second amount of the second therapeutic agent is chosen to elute a therapeutic dosage of the respective therapeutic agent over a period of time.

14. The hemodialysis graft of claim 13, wherein:
each of the first coating deposit and the second coating deposit comprises a non-bioerodible polymer in which the first therapeutic agent and the second therapeutic agent is mixed, respectively.

15. The hemodialysis graft of claim 1, further comprising:
a third coating deposit located on the first coating deposit; and
a fourth coating deposit located on the second coating deposit,
wherein the third coating deposit comprises a third therapeutic agent and the fourth coating deposit comprises a fourth therapeutic agent.

16. The hemodialysis graft of claim 15, wherein at least one of the third and fourth therapeutic agents has anti-stenotic properties.

17. The hemodialysis graft of claim 16, wherein:
at least one of the first coating deposit and the second coating deposit comprises a nonbioerodible polymer; and
at least one of the third and fourth coating deposits comprises a bioerodible polymer.

18. A graft, comprising:
a substantially cylindrical and hollow tube of a predetermined longitudinal length having a distal end with a distal opening, a proximal end with a proximal opening, an interior surface and an exterior surface;
a first coating deposit located substantially only on the interior surface at the distal end and extending proximally and longitudinally a first predetermined distance; and
a second coating deposit located substantially only on the exterior surface at the distal end and extending proximally and longitudinally a second predetermined distance,
wherein the first coating deposit comprises a first therapeutic agent and the second coating deposit comprises a second therapeutic agent, and
wherein the tube comprises a substantially impermeable material.

19. The graft of claim 18, wherein the first and second therapeutic agents are different from one another.

20. The hemodialysis graft of claim 18, wherein at least one of the first and second therapeutic agents has anti-stenotic properties.

21. The hemodialysis graft of claim 18, wherein:
the first therapeutic agent is one of: paclitaxel, a paclitaxel derivative, sirolimus, an Interleukin-2 inhibitor, dexamethasone, a steroid, dopidogel, a platelet aggregate inhibitor, and fish oil; and
the second therapeutic agent is one of paclitaxel, a paclitaxel derivative, sirolimus, an Interleukin-2 inhibitor, dexamethasone, a steroid, dopidogel, a platelet aggregate inhibitor, and fish oil.

22. The graft of claim 18, wherein the first coating deposit further comprises a third therapeutic agent and the second coating deposit further comprises a fourth therapeutic agent.

23. The graft of claim 18, wherein the tube comprises at least one of:
expanded polytetrafluoroethylene (ePTFE);
polyvinylchloride polypropylene;
fluorinated ethylene propylene;
polyetherurethaneurea; and
a biocompatible plastics material.

24. The graft of claim 18, wherein:
the interior surface comprises a third therapeutic agent disposed in a polymer.

25. The graft of claim 24, wherein the polymer is one of: a bio-erodible type and a non-bioerodible type.

26. A method of making an asymmetric drug eluting hemodialysis graft, the method comprising:
providing a substantially cylindrical and hollow tube of a substantially impermeable material, the tube having a predetermined longitudinal length, a distal end with a distal opening, a proximal end with a proximal opening, an interior surface and an exterior surface;
applying a first coating substantially only on the interior surface at the distal end and extending proximally and longitudinally a first predetermined distance, the first coating comprising a first therapeutic agent;
applying a second coating substantially only on the exterior surface at the distal end and extending proximally and longitudinally a second predetermined distance, the second coating comprising a first therapeutic agent; and
applying the first and second coating such that substantially none of the first and second coating is applied at the interior and exterior surfaces of the proximal end of the tube.

27. The method of claim 26, wherein the tube comprises at least one of:
expanded polytetrafluoroethylene (ePTFE);
polyvinylchloride polypropylene;
fluorinated ethylene propylene;
polyetherurethaneurea; and
a biocompatible plastics material.

28. The method of claim 26, further comprising:
providing a first and second anti-stenotic, respectively, for the first and second therapeutic agents.

29. The method of claim of claim 28, further comprising:
choosing the first and second anti-stenotic from the group comprising: paclitaxel, a paclitaxel derivative, sirolimus, an Interleukin-2 inhibitor, dexamethasone, a steroid, dopidogel, a platelet aggregate inhibitor, and fish oil.

30. A method, comprising:
providing a hemodialysis graft, the hemodialysis graft comprising:
a distal end, a proximal end, an interior surface and an exterior surface;
a first coating deposit located substantially only on the interior surface at the distal end and extending proximally and longitudinally a first predetermined distance, the first coating deposit comprising a first therapeutic agent; and
a second coating deposit located substantially only on the exterior surface at the distal end and extending proximally and longitudinally a second predetermined distance, the second coating deposit comprising a second therapeutic agent;
attaching the distal end of the hemodialysis graft to a vein of a patient; and attaching the proximal end of the hemodialysis graft to an artery of the patient,
wherein at least one of the first and second therapeutic agents elutes from the respective coating deposit.
